(19) United States
(12) Patent Application Publication
(10) Pub. No.: US 2025/0359843 A1
(43) Pub. Date: Nov. 27, 2025

(54) LUNG FUNCTION TESTING

(71) Applicant: EUPNOOS LTD, London (GB)

(72) Inventors: Arshia GRATIOT, London (GB); Chas SHEPPARD, London (GB); Mahdi SHABAN, London (GB)

(73) Assignee: EUPNOOS LTD, London (GB)

(21) Appl. No.: 18/872,484

(22) PCT Filed: Jun. 7, 2023
(86) PCT No.: PCT/GB2023/051487

§ 371 Date: Dec. 6, 2024

(30) Foreign Application Priority Data
Jun. 7, 2022 (GB) 2208360.4

(51) Int. Cl. A61B 7/04; A61B 5/00; A61B 5/107; G06V 40/16

(52) U.S. Cl. CPC A61B 7/04; A61B 5/1077; A61B 5/1079; A61B 5/7425; G06V 40/166; G06V 40/171

(57) ABSTRACT

Methods are disclosed for determining an optimal distance between an electronic device and a user providing an audio sample of a respiratory manoeuvre for use in assessing the lung function of the user. An audio sample dataset comprising a plurality of audio samples of respiratory manoeuvres performed by the user at a plurality of distances between the electronic device and the user is received from an audio sensor in the electronic device. The audio sample dataset is analysed to determine an optimum audio sample of a respiratory manoeuvre for use in assessing the lung function of the user. The optimum distance corresponding with the optimum audio sample is determined. The user is instructed to place the electronic device within a threshold distance of the optimum distance and perform a respiratory manoeuvre. Audio samples of the respiratory manoeuvre at the optimum distance can be used in assessing user lung function.

### FIELD OF THE INVENTION

The invention relates to methods for optimising lung function testing on an electronic device. In particular, the invention relates to capturing an optimum audio sample of a respiratory manoeuvre for use in assessing the lung function of the user. Specifically, the invention relates to capturing an optimum audio sample while the user is providing an optimum open mouth shape during the respiratory manoeuvre, and capturing an optimum audio sample when the electronic device is located at an optimum distance from the user's mouth.

### BACKGROUND OF THE INVENTION

Respiratory diseases such as chronic obstructive pulmonary disease (COPD), emphysema, chronic bronchitis, asthma, cystic fibrosis (CF), and interstitial lung disease (ILD) are a major cause of societal, health, and economic burdens worldwide.

The most common clinical pathways for the identification and diagnosis of respiratory disease are via the application of quality assured pulmonary function testing (PFT). Typically, a procedure is carried out in-clinic via a specialised device called a spirometer that involves a user performing a respiratory manoeuvre by forcedly exhaling into the end of a cylindrical tube multiple times under the guidance and coaching of a clinician.

According to established clinical guidelines for spirometry, lung vital capacity (LVC) is determined by assessing the volume of air that the patient can expel from the lungs after a maximal inspiration (FVC), or maximum expiration in one second (FEV1). It is a reliable method of differentiating between obstructive airways disorders (e.g., chronic obstructive pulmonary disease, asthma) and restrictive diseases (e.g. fibrotic lung disease). Aside from being used to classify lung conditions into obstructive or restrictive patterns, it can also help to monitor exacerbation of symptoms and disease severity. While spirometry alone cannot establish a diagnosis of a specific disease, it is sufficiently reproducible to be useful in following the course of many different diseases if done correctly.

With the increasing adoption of virtual healthcare assessment and home management of chronic conditions, it is desirable to enable remote lung function testing and monitoring at home. However, clinical spirometry devices have many limitations which make them unsuitable for home use, such as availability, usability, size and cost. Whilst portable spirometry devices exist, for example, connecting to a smartphone via Bluetooth to enable a user to perform PFT away from a clinical setting, the absence of guidance and coaching from a clinician can lead to inconsistent and unreliable results should the user not perform the respiratory manoeuvre correctly and/or repeatably.

Another existing approach to remote lung function testing and monitoring that obviates the need for a clinical spirometer involves using the microphone of a smartphone held roughly at arm's length to record a respiratory manoeuvre performed by a user. However, this existing approach has a drawback that inconsistencies in the recording of the respiratory manoeuvre at the smartphone microphone may occur, leading to the risk of subsequent misdiagnoses by a clinician interpreting the results recorded by the smartphone.

Therefore, it would be desirable to provide a way of enabling a user to produce accurate and repeatable spirometry data in a non-clinical setting.

### SUMMARY OF THE INVENTION

According to a first aspect of the invention, there is provided a computer-implemented method of obtaining an audio sample of a respiratory manoeuvre for use in assessing the lung function of a user. The computer-implemented method comprises the steps of: determining a minimum area of an open mouth of the user required for providing an optimum audio sample of a respiratory manoeuvre for use in assessing the lung function of the user; receiving, from a user-facing camera in an electronic device, image data of the user's face including at least an open mouth of the user; identifying, from the image data, an area defined by the open mouth; determining whether the area of the open mouth from the image data is at least equal to the minimum area of the open mouth required for providing the optimum audio sample of the respiratory manoeuvre; and guiding the user to achieve the optimum mouth shape. Guiding the user comprises providing an indication whether the area of the open mouth of the user is at least equal to the minimum area of the open mouth required for providing the audio sample of the respiratory manoeuvre.

In this way, a user is able to provide accurate and repeatable out-of-clinic audio samples of respiratory manoeuvres for use in user lung function assessment without the need for mouthpieces or tubes associated with conventional spirometry data collection. In other words, the user needs only their electronic device (such as a smartphone) to provide audio samples; no clinician or specialist equipment is required.

The computer-implemented method may further comprise determining, from the image data, that the area of the open mouth is at least equal to the minimum area of the open mouth required for providing the audio sample of the respiratory manoeuvre, instructing the user to perform a respiratory manoeuvre, and receiving, from an audio sensor in the electronic device, an audio sample of the respiratory manoeuvre.

The computer-implemented method may further comprise receiving, from the user-facing camera in the electronic device, further image data of the user's face including at least the open mouth of the user as the user performs the respiratory manoeuvre, and determining whether the minimum area of the open mouth is maintained for at least an optimum period of the audio sample. In this way, a reliable audio sample may be obtained across the entire optimum period of the respiratory manoeuvre. In other words, if the user fails to keep their mouth open to at least the minimum area during the optimum period of the respiratory manoeuvre, the audio sample may be unreliable for assessing the lung function of the user and the user may be prompted to repeat the respiratory manoeuvre.

The indication may provide a prompt to the user on the electronic device. The prompt may comprise a visual or audio cue for indicating to the user whether the minimum area of the open mouth required for providing the audio sample of the respiratory manoeuvre is achieved. Alternatively, the indication may provide an overlay on the image data displayed on the electronic device, where the overlay may be based on the minimum area of the open mouth required for providing the audio sample of the respiratory manoeuvre. In this way, the user is guided to open their mouth to the minimum required area, thus ensuring that the audio sample of the respiratory manoeuvre subsequently performed by the user is devoid of airflow restriction artefacts.

The minimum area of the mouth may be related to an expected area of the throat of the user, for example, the minimum area of the mouth may be greater than the expected area of the throat of the user. In this way, restriction to the airflow produced as the user performs a respiratory manoeuvre is avoided, thus reducing the risk of artefacts in the audio sample.

The area defined by the open mouth in the image may be determined by segmenting the image into an open-mouth part and a non-open-mouth part by thresholding the image according to the relative brightness of the open-mouth part and the non-open-mouth part.

Pixels in the image that are associated with the open-mouth part are expected to be darker than pixels associated with the non-open-mouth parts.

The area defined by the open mouth in the image may be determined by locating eyes in the image data and identifying the open mouth relative to the location of the eyes.

The computer-implemented method may further comprise receiving, from the user-facing camera in the electronic device, further image data of the user's face including at least the open mouth of the user as the user performs the respiratory manoeuvre. From the further image data, a size of the user's head may be identified. By looking for changes in the size of the user's head between successive frames of the further image data, it may be determined if the user is moving their head towards, or away from, the electronic device. Based on this determination, it may then be determined if the respiratory manoeuvre is an expected respiratory manoeuvre. For example, if an exhalation was expected, but the user is determined to have performed an inhalation, then the audio sample can be discarded, and the user prompted to repeat the respiratory manoeuvre.

According to a second aspect of the invention, there is provided a computer readable medium comprising instructions that, when executed by a processor, cause the processor to carry out the method of the first aspect of the invention.

According to a third aspect of the invention, there is provided a computer-implemented method of determining an optimum distance between an electronic device and a user providing an audio sample of a respiratory manoeuvre for use in assessing the lung function of the user. The computer-implemented method comprises receiving, from an audio sensor in the electronic device, an audio sample dataset comprising a plurality of audio samples of respiratory manoeuvres performed by the user at a plurality of distances between the electronic device and the user. The audio sample dataset comprises at least one audio sample of a respiratory manoeuvre for each of the plurality of distances. The method further involves determining an optimum distance for a user to provide an optimum audio sample of a respiratory manoeuvre for use in assessing the lung function of the user based on the audio sample dataset.

In this way, a user-specific optimum smartphone-to-mouth distance can be determined. As such, when performing subsequent respiratory manoeuvres, the user is able to position their smartphone at the particular optimum distance for them. As a result, the accuracy of lung function data obtained is not adversely affected by variances in the position a user holds their smartphone, or the amount of force with which they perform the respiratory manoeuvre, which might vary between measurements taken at different times. Also, the claimed technique avoids the need to make assumptions about the length of a users' arms and the amount of force with which different users are able to perform respiratory manoeuvres which could adversely affect the accuracy of lung function data obtained. This allows for accurate and repeatable lung function testing in a non-clinic setting, and the risk of misdiagnoses is reduced.

The optimum distance may be determined based on a signal-to-noise ratio and a distortion level for each of the plurality of audio samples in the audio sample dataset.

The optimum distance may correspond with an audio sample in the audio sample dataset having a signal-to-noise ratio above a noise threshold and/or a distortion level below a distortion threshold. In this way, audio samples that do not meet the noise and distortion threshold criteria may be discarded, thus reducing the risk of a clinician making any diagnosis based on audio samples containing artefacts (low signal-to-noise ratio and/or high distortion levels).

The optimum distance may correspond with a predicted optimum audio sample based on the audio sample dataset. The predicted optimum audio sample may be determined by fitting a function to the signal-to-noise ratio and/or distortion level of the audio sample dataset in order to predict an optimum audio sample where the signal-to-noise ratio is maximised, and the distortion level is minimised. This “fitting” approach can be useful when the number of audio samples in the audio sample dataset is relatively low, as the “true” optimum audio sample (and corresponding “true” optimum distance) is more likely to fall somewhere between neighbouring audio samples.

Determining the signal-to-noise ratio may comprise receiving, from an audio sensor in the electronic device, background audio data relating to background noise of the environment of the user and comparing each audio sample with the background audio data. In this way, the effect of the background noise level on each audio sample can be assessed to determine if, for example, the background noise is at a level that would affect the ability to discern the audio signal of the respiratory manoeuvre from the background noise in the environment of the user.

The computer-implemented method may further comprise receiving, from a user-facing camera in the electronic device, image data of the user's face corresponding with each of the audio samples. A feature of the user's face may be extracted from the image data associated with each of the audio samples. A distance between the user and the electronic device of each audio sample may be determined based on the feature extracted from the image data associated with the respective audio sample. This provides a way to reference the optimum distance between the user and the electronic device so that the optimum distance can reliably be found again during future lung function measurements. Using facial features as a reference removes any subjectivity (i.e., does not rely on a user holding the smartphone at their perceived arm's length which might vary from time-to-time) improving accuracy/reliability between lung function measurements made at different times.

The feature may be a distance between the eyes of the user.

The distortion level may be based on the level of one or more of non-linear distortion, windshear, and clipping in an audio sample.

The respiratory manoeuvre may be an inspiratory manoeuvre or an expiratory manoeuvre. The optimum distance for an inspiratory manoeuvre may be different to the optimal distance for an expiratory manoeuvre. By determining that an inspiratory manoeuvre has a different optimum distance than an expiratory manoeuvre, the quality of a recorded audio sample is improved. For example, since inspiratory manoeuvres are typically audibly quieter than expiratory manoeuvres, the user can be encouraged to position the electronic device at a distance closer to their mouth. Conversely, for expiratory manoeuvres the user can be encouraged to position the electronic device comparatively further from their mouth, thus avoiding detrimental audio artefacts such as windshear and clipping.

According to a fourth aspect of the invention, there is provided a computer readable medium comprising instructions that, when executed by a processor, cause the processor to carry out the method of the third aspect of the invention.

According to a fifth aspect of the invention, there is provided a computer-implemented method of recording an audio sample of a respiratory manoeuvre performed by a user for use in assessing the lung function of the user. The computer-implemented method comprises the steps of: receiving, from an audio sensor in the electronic device, an audio sample dataset comprising a plurality of audio samples of respiratory manoeuvres performed by the user at a plurality of distances between the electronic device and the user, wherein the audio sample dataset comprises at least one audio sample of a respiratory manoeuvre for each of the plurality of distances; determining an optimum distance for a user to provide an optimum audio sample of a respiratory manoeuvre for use in assessing the lung function of the user based on the audio sample dataset; instructing the user to position the electronic device such that a distance between the user and the electronic device is within a threshold distance of the optimum distance and, upon determining that the distance between the user and the electronic device is within the threshold distance of the optimum distance, instructing the user to perform a respiratory manoeuvre; and receiving, from an audio sensor in the electronic device, an audio sample of the respiratory manoeuvre.

By instructing the user to position the electronic device at the optimum distance and determining that the distance between the user and the electronic device is within a threshold distance of the optimum distance, accurate and repeatable lung function data can be obtained from the user each time the user performs a respiratory manoeuvre, reducing the risk of misdiagnoses and allowing for lung function data to be compared over time more reliably.

Determining the distance between the user and the electronic device may comprise receiving, from a user-facing camera in the electronic device, image data of the user's face. A feature of the user's face may be extracted from the image data. The distance between the user and the electronic device may be determined based on the feature extracted from the image data. This provides a way to reference the optimum distance between the user and the electronic device so that the optimum distance can reliably be found again during future lung function measurements. Using facial features as a reference removes any subjectivity (i.e., does not rely on a user holding the smartphone at their perceived arm's length which might vary from time-to-time) improving accuracy/reliability between measurements made at different times.

The feature may comprise a distance between the eyes of the user.

The respiratory manoeuvre may be an inspiratory manoeuvre or an expiratory manoeuvre. By having a different optimal distance for an inspiratory manoeuvre than an expiratory manoeuvre, the quality of a recorded audio sample for determining lung function is improved. For example, since inspiratory manoeuvres are typically audibly quieter than expiratory manoeuvres, the user can be encouraged to position the electronic device at a distance closer to their mouth, improving signal-to-noise. Conversely, for expiratory manoeuvres the user can be encouraged to position the electronic device comparatively further from their mouth, thus avoiding detrimental audio artefacts such as windshear and clipping.

According to a sixth aspect of the invention, there is provided a computer readable medium comprising instructions that, when executed by a processor, cause the processor to carry out the method of any of the fifth aspect of the invention.

### FURTHER ASPECTS OF THE INVENTION

According to a seventh aspect of the invention, there is provided a computer-implemented method of determining an optimal distance between an electronic device and a user providing an audio sample of a respiratory manoeuvre for use in assessing the lung function of the user. The computer-implemented method comprises the steps of: receiving, from an audio sensor in the electronic device, an audio sample dataset comprising a plurality of audio samples of respiratory manoeuvres performed by the user at a plurality of distances between the electronic device and the user, wherein the audio sample dataset comprises at least one audio sample of a respiratory manoeuvre for each of the plurality of distances; analysing the audio sample dataset to determine an optimum audio sample of a respiratory manoeuvre for use in assessing the lung function of the user; and determining the optimum distance corresponding with the optimum audio sample.

By analysing the audio sample dataset to determine an optimum audio sample and determining the optimum distance corresponding with the optimum audio sample, a user-specific optimum smartphone-to-mouth distance is determined. As such, when performing subsequent respiratory manoeuvres, the user is able to position their smartphone at the particular optimum distance for them. As a result, the accuracy of lung function data obtained is not adversely affected by variances in the position a user holds their smartphone, or the amount of force with which they perform the respiratory manoeuvre, which might vary between measurements taken at different times. Also, the claimed technique avoids the need to make assumptions about the length of a users' arms and the amount of force with which different users are able to perform respiratory manoeuvres which could adversely affect the accuracy of lung function data obtained. This allows for accurate and repeatable lung function testing in a non-clinic setting, and the risk of misdiagnoses is reduced.

Analysing the audio sample dataset may comprise determining a signal-to-noise ratio and a distortion level for each of the plurality of audio samples. The optimum audio sample may be determined based on an audio sample of the plurality of audio samples having a signal-to-noise ratio above a noise threshold and a distortion level below a distortion threshold. In this way, audio samples that do not meet the noise and distortion threshold criteria may be discarded, thus reducing the risk of a clinician making any diagnosis based on audio samples containing artefacts (low signal-to-noise ratio and/or high distortion levels).

Determining the signal-to-noise ratio may comprise receiving, from an audio sensor in the electronic device, background audio data relating to background noise of the environment of the user and comparing each audio sample with the background audio data. In this way, the effect of the background noise level on each audio sample can be assessed to determine if, for example, the background noise is at a level that would affect the ability to discern the audio signal of the respiratory manoeuvre from the background noise in the environment of the user.

The computer-implemented method may further comprise receiving, from a user-facing camera in the electronic device, image data of the user's face corresponding with each of the audio samples. A feature of the user's face may be extracted from the image data associated with each of the audio samples. A distance between the user and the electronic device of each audio sample may be determined based on the feature extracted from the image data associated with the respective audio sample. This provides a way to reference the optimum distance between the user and the electronic device so that the optimum distance can reliably be found again during future lung function measurements. Using facial features as a reference removes any subjectivity (i.e., does not rely on a user holding the smartphone at their perceived arm's length which might vary from time-to-time) improving accuracy/reliability between lung function measurements made at different times.

The feature may comprise a distance between the eyes of the user.

The distortion level may be based on the level of one or more of non-linear distortion, windshear, and clipping in an audio sample.

The respiratory manoeuvre may be an inspiratory manoeuvre or an expiratory manoeuvre. The optimum distance for an inspiratory manoeuvre may be different to the optimal distance for an expiratory manoeuvre. By determining that an inspiratory manoeuvre has a different optimum distance than an expiratory manoeuvre, the quality of a recorded audio sample is improved. For example, since inspiratory manoeuvres are typically audibly quieter than expiratory manoeuvres, the user can be encouraged to position the electronic device at a distance closer to their mouth. Conversely, for expiratory manoeuvres the user can be encouraged to position the electronic device comparatively further from their mouth, thus avoiding detrimental audio artefacts such as windshear and clipping.

According to an eighth aspect of the invention, there is provided a computer readable medium comprising instructions that, when executed by a processor, cause the processor to carry out the computer implemented method of the seventh aspect of the invention.

According to a ninth aspect of the invention, there is provided a computer-implemented method of recording an audio sample of a respiratory manoeuvre performed by a user for use in assessing the lung function of the user. The computer-implemented method comprises retrieving an optimum distance between an electronic device and the user for providing an audio sample of a respiratory manoeuvre for use in assessing the lung function of the user. The optimum distance may be determined using the method according to the first aspect. The method further comprises instructing the user to position the electronic device such that a distance between the user and the electronic device is within a threshold distance of the optimum distance and, upon determining that the distance between the user and the electronic device is within the threshold distance of the optimum distance, instructing the user to perform a respiratory manoeuvre; and receiving, from an audio sensor in the electronic device, an audio sample of the respiratory manoeuvre.

By instructing the user to position the electronic device at the optimum distance and determining that the distance between the user and the electronic device is within a threshold distance of the optimum distance, accurate and repeatable lung function data can be obtained from the user each time the user performs a respiratory manoeuvre, reducing the risk of misdiagnoses and allowing for lung function data to be compared over time more reliably.

Determining the distance between the user and the electronic device may comprise receiving, from a user-facing camera in the electronic device, image data of the user's face. A feature of the user's face may be extracted from the image data. The distance between the user and the electronic device may be determined based on the feature extracted from the image data. This provides a way to reference the optimum distance between the user and the electronic device so that the optimum distance can reliably be found again during future lung function measurements. Using facial features as a reference removes any subjectivity (i.e., does not rely on a user holding the smartphone at their perceived arm's length which might vary from time-to-time) improving accuracy/reliability between measurements made at different times.

The feature may comprise a distance between the eyes of the user.

The respiratory manoeuvre may be an inspiratory manoeuvre or an expiratory manoeuvre. The optimum distance for an inspiratory manoeuvre may be different to the optimal distance for an expiratory manoeuvre. By having a different optimal distance for an inspiratory manoeuvre than an expiratory manoeuvre, the quality of a recorded audio sample for determining lung function is improved. For example, since inspiratory manoeuvres are typically audibly quieter than expiratory manoeuvres, the user can be encouraged to position the electronic device at a distance closer to their mouth, improving signal-to-noise. Conversely, for expiratory manoeuvres the user can be encouraged to position the electronic device comparatively further from their mouth, thus avoiding detrimental audio artefacts such as windshear and clipping.

According to a tenth aspect of the invention, there is provided a computer readable medium comprising instructions that, when executed by a processor, cause the processor to carry out the method according to the ninth aspect of the invention.

According to a eleventh aspect of the invention, there is provided a computer-implemented method of determining an optimum open mouth shape of a user providing an audio sample of a respiratory manoeuvre for use in assessing the lung function of the user. The user may first position the electronic device at the optimum distance determined using the method according to the first aspect. The computer-implemented method of determining an optimum open mouth shape comprises determining a minimum area of the open mouth of the user required for providing an optimum audio sample of a respiratory manoeuvre for use in assessing the lung function of the user; receiving, from a user-facing camera in an electronic device, image data of the user's face including at least an open mouth of the user; identifying, from the image data, an area defined by the open mouth; and determining whether the area of the open mouth from the image data is at least equal to the minimum area of the open mouth required for providing the audio sample of the respiratory manoeuvre.

In this way, a user is able to provide accurate and repeatable out-of-clinic audio samples of respiratory manoeuvres for use in user lung function assessment without the need for mouthpieces or tubes associated with conventional spirometry data collection. In other words, the user needs only their electronic device (such as a smartphone) to provide audio samples; no clinician or specialist equipment is required.

The minimum area of the mouth may be related to an expected area of the throat of the user, for example, the minimum area of the mouth may be greater than the expected area of the throat of the user. In this way, restriction to the airflow produced as the user performs a respiratory manoeuvre is avoided, thus reducing the risk of artefacts in the audio sample.

The expected area of the throat may be determined based on one of more characteristics of the user, such as age, gender and ethnicity, which allow the expected area of the throat to be estimated.

The area defined by the open mouth in the image is determined by segmenting the image into an open-mouth part and a non-open-mouth part. Segmenting the image may comprises thresholding the image according to the relative brightness of the open-mouth part and the non-open-mouth part. Pixels in the image that are associated with the open-mouth part are expected to be darker than pixels associated with the non-open-mouth parts.

The area defined by the open mouth in the image may be determined by locating eyes in the image data and identifying the open mouth relative to the location of the eyes.

The computer-implemented method may further comprise guiding the user to achieve the optimum open mouth shape. Guiding the user may comprise providing an overlay on the image data displayed on the electronic device. The overlay may be based on the minimum area of the open mouth required for providing the audio sample of the respiratory manoeuvre. In this way, the user is guided to open their mouth to the minimum required area, thus ensuring that the audio sample of the respiratory manoeuvre subsequently performed by the user is devoid of airflow restriction artefacts.

The computer-implemented method may further comprise, upon determination that the area of the open mouth from the image data is at least equal to the minimum area of the open mouth required for providing the audio sample of the respiratory manoeuvre, instructing the user to perform a respiratory manoeuvre. An audio sensor in the electronic device then receives an audio sample of the respiratory manoeuvre performed by the user. In this way, a reliable audio sample can be received, as the user is only instructed to perform the respiratory manoeuvre upon determination that the area of the open mouth from the image data is at least equal to the minimum area of the open mouth required for providing the audio sample of the respiratory manoeuvre. Additionally, the user needs only their electronic device (such as a smartphone) to understand whether they have opened their mouth sufficiently to provide an audio sample of a respiratory manoeuvre; no clinician or specialist equipment is required.

According to a twelfth aspect of the invention, there is provided a computer readable medium, comprising instructions that when executed by a processor, cause the processor to carry out the method of the eleventh aspect of the invention.

### BRIEF DESCRIPTION OF THE DRAWINGS

The invention will now be described, by way of example only, with reference to the accompanying drawings, in which:

FIG. 1 is a diagram illustrating a smartphone positioned at a distance from a mouth of a user;

FIG. 2 is a diagram illustrating a smartphone positioned at a number of distances from a mouth of a user in order to determine an optimum distance from the mouth of the user to the smartphone;

FIG. 3 is flowchart illustrating a method for determining the optimum distance for positioning a smartphone from a mouth of a user based on an audio sample dataset;

FIG. 4 is a diagram illustrating a method for recording an audio sample of a respiratory manoeuvre performed at the optimum distance;

FIG. 5 is a diagram illustrating a smartphone running a user interface for guiding the user to position their smartphone at the optimum distance;

FIG. 6 is a flowchart illustrating a method of determining an optimum open mouth shape of a user providing an audio sample of a respiratory manoeuvre for use in assessing the lung function of the user; and

FIGS. 7 and 8 are diagrams of a smartphone having a user interface for guiding a user in providing an optimum mouth shape for providing an audio sample of a respiratory manoeuvre.

### DETAILED DESCRIPTION

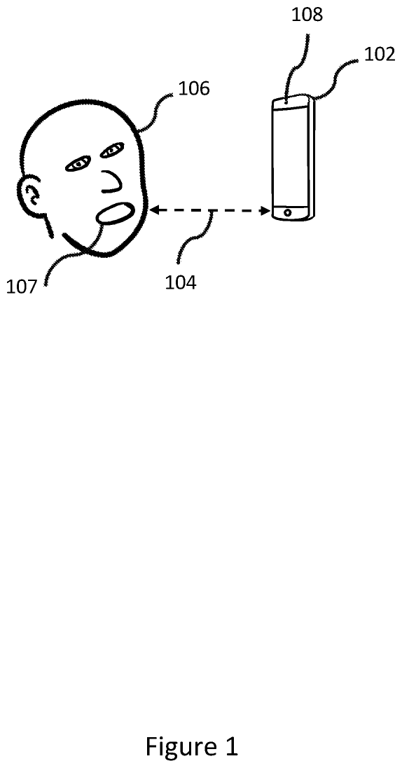

FIG. 1 illustrates a user 106 using a smartphone 102 to make a lung function measurement. The user 106 typically holds the smartphone 102 in their hand at a distance 104 from their mouth 107 which is approximately arm's length from the user 106. The user 106 performs a respiratory manoeuvre that is recorded by a microphone in the smartphone 102 and stored as an audio sample on the smartphone 102. The audio sample is processed (either on the smartphone 102 or at a remote server) to determine one or more aspects of the user's lung function from the audio sample.

The respiratory manoeuvre performed by the user 106 is either an expiratory manoeuvre or an inspiratory manoeuvre.

Prior to an expiratory manoeuvre, the user 106 generally performs a deep inspiration where the user 106 tries to fill their lungs to capacity. The user 106 then performs an expiratory manoeuvre in which the user 106 exhales towards the smartphone 102 in an attempt to empty their lungs completely. An audio sample of the expiratory manoeuvre is recorded by the smartphone 102 and can be analysed to determine various aspects of the user's lung function, such as: the forced expiratory volume of the user 106 in one second, FEV1 (i.e., the volume of air exhaled by the user in the first second following deep inspiration and forced expiration); the forced vital capacity, FVC (i.e., the total volume of air that the user can forcibly exhale in one breath); the slow vital capacity, SVC (i.e., the volume of air exhaled by the used in an unforced manoeuvre); among others.
Prior to an inspiratory manoeuvre, the user 106 generally performs a deep expiration where the user 106 tries to fully empty their lungs. The user 106 then performs an inspiratory manoeuvre which involves the user 106 inhaling in the direction of the smartphone 102 to record an audio sample of the inspiratory manoeuvre. Performing the inspiratory manoeuvre typically requires the user 106 to position the smartphone 102 much closer to their mouth 107 than when performing an expiratory manoeuvre since an inspiratory manoeuvre is typically much quieter. The audio sample of the inspiratory manoeuvre can subsequently be used to determine aspects of the user's lung function, such as the inspiratory vital capacity, IVC (i.e., the volume of air that can be inspired following a normal expiration).

### Guiding a User on the Optimum Distance for a Respiratory Manoeuvre

Without guiding the user 106 about how far they should hold the smartphone 102 from their mouth 107 while performing a respiratory manoeuvre, there tends to be variation in the distance 104 according to the length of the arm of the user 106 (which will naturally vary according to the age and gender of the user 106). Additionally, whether the arm of the user 106 is fully extended or slightly flexed each time they perform a respiratory manoeuvre, leads to variations in the audio samples of the respiratory manoeuvres that are recorded, which can lead to errors in the lung function measurements.

There is also variability in the amount of force with which different users can perform respiratory manoeuvres meaning that an arm's length distance may not be appropriate for all users. For example, one user may perform a respiratory manoeuvre particularly forcefully, resulting in windshear defects at the smartphone microphone (i.e., the distance 104 needs to be increased for that user to eliminate such windshear defects). Conversely, another user may be unable to perform a respiratory manoeuvre with enough force to produce sufficient sound pressure such that the smartphone microphone is able to detect the respiratory manoeuvre if the smartphone is held at arm's length (i.e., the distance 104 needs to be reduced for that user).

There is also variability in the level of force with which different users (or for that matter, the same user) may perform a respiratory manoeuvre as their lung condition changes (worsens or improves). This can lead to errors in the lung function measurements which result in misdiagnoses by a clinician interpreting the lung function measurements.

Additionally, owing to the fact that inspiratory manoeuvres are typically audibly quieter than expiratory manoeuvres, holding the smartphone 102 at arm's length may not be suitable for recording an inspiratory manoeuvre as the volume thereof may be below a reliable detection sensitivity of the smartphone microphone.

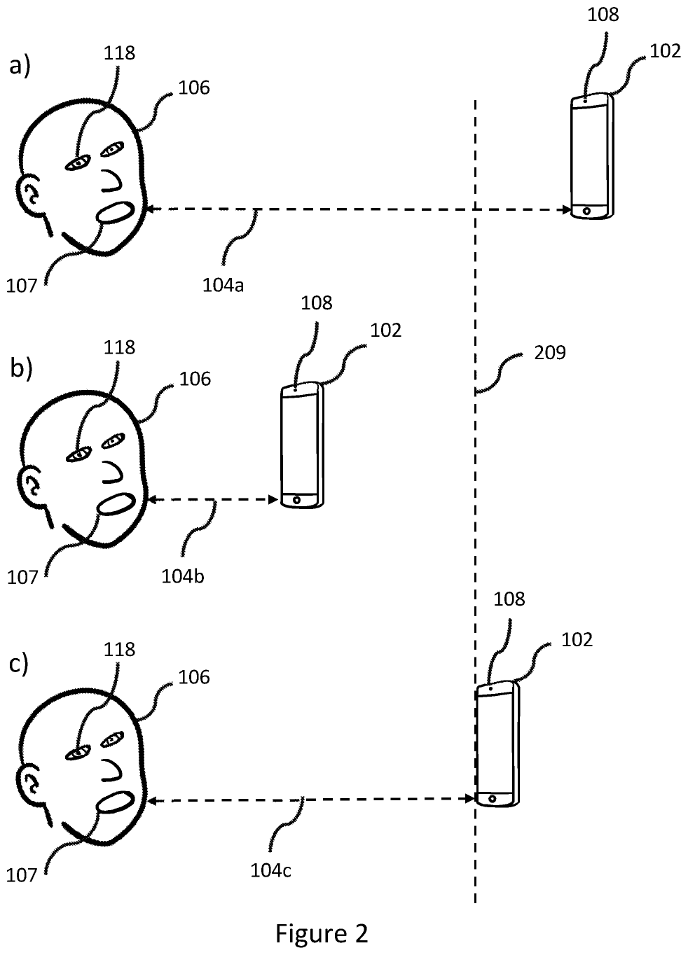

FIG. 2 illustrates a method for identifying an optimum distance 209 at which the smartphone 102 should be positioned from the mouth 107 of the user 106 to record an optimum audio sample by instructing the user 106 to place the smartphone at a number of different distances 104a-104c away from their mouth 107:
Initially, the user 106 is instructed to hold the smartphone 102 at a relatively long distance 104a from their mouth 107, for example, at a maximum arm's length with the user's arm fully extended away from their mouth 107 (FIG. 2a). A front-facing camera 108 in the smartphone 102 captures an image of the user 106 at distance 104a. The user 106 is then instructed to perform a respiratory manoeuvre.
Next, the user 106 is instructed to hold the smartphone 102 at a relatively close distance 104b to their mouth 107 (FIG. 2b). Again, the camera 108 captures an image of the user 106 at distance 104b. The user 106 is then instructed to perform a respiratory manoeuvre.
Then, the user 106 is instructed to hold the smartphone 102 at one or more intermediate distances 104c from their mouth 107 (FIG. 2c) and the camera 108 captures an image of the user 106 at each of the intermediate distances 104c. The user 106 is then instructed to perform a respiratory manoeuvre at each intermediate distance 104c.

An audio sample of the respiratory manoeuvre performed by the user 106 is recorded on the smartphone 102 at each of the distances 104a-104c to form an audio sample dataset.

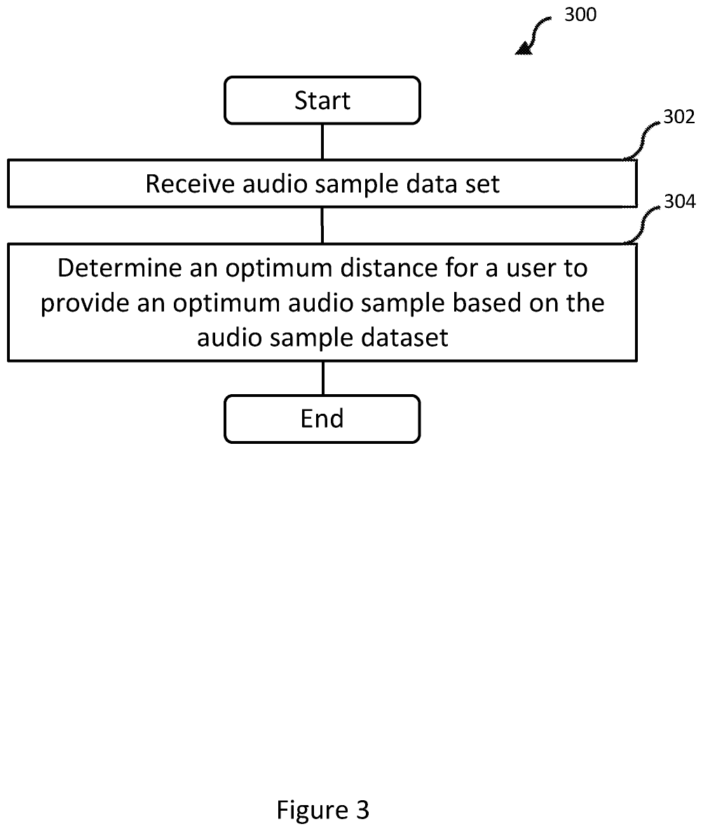

FIG. 3 is a flowchart 300 illustrating a method for determining an optimum distance from the audio sample dataset. At step 302, the audio sample dataset comprising the plurality of audio samples of respiratory manoeuvres performed by the user 106 holding the smartphone 102 at the plurality of distances from their mouth 107, as described above with respect to FIGS. 2a-2c, is received at the smartphone 102 or at a server for processing.

At step 304 the audio sample dataset is processed, at the smartphone 102 or on a server, to determine an optimum distance for a user to provide an optimum audio sample for use in assessing the lung function of the user 106 (i.e., the optimum distance is a distance between the user's smartphone 102 and the user's mouth 107 that allows for an optimum audio sample for assessing the lung function of the user 106 to be recorded). A distance between the smartphone 102 and the user's mouth 107 may be determined for each audio sample in the audio sample dataset. As outlined above with respect to FIGS. 2a-2c, an image of the user 106 is captured by the camera 108 along with each audio sample. This image will show facial features of the user 106 that can be used to determine a distance 209 between the camera 108 and the user 106. For example, a physical spacing between a user's eyes 118 is well-characterised for people of a particular age, gender and ethnicity. The pixel spacing between the facial features of the user 106 in the image associated with the optimum audio sample can be determined. From a knowledge of the focal length of the lens of the camera 108, the distance d can be determined according to the relationship: d=(W*f)/w, where: W is the physical distance between the user's eyes 118 (determined by looking up a known value based on the age, gender and ethnicity of the user 106); w is the pixel space between the eyes 118 of the user 106 in the image associated with the optimum audio sample; and f is the focal length of the lens of the camera 108.

A signal-to-noise ratio and/or a signal distortion level may be determined for each of the audio samples in the audio sample dataset using any suitable signal processing technique known to the skilled person. The signal-to-noise ratio relates to a ratio of the signal relating to the respiratory manoeuvre performed by the user 106 and a background noise level of the environment of the user 106 during a time period in which the user 106 performs the respiratory manoeuvre. The signal distortion level relates to a measure of audio defects in each audio sample such as non-linear distortion, windshear, and clipping in each audio sample.

An optimum audio sample can be determined as an audio sample that will meet the condition of having a signal-to-noise ratio above a noise threshold and/or a distortion level below a distortion threshold. An extreme distortion level could masquerade itself as an unwanted lung condition (for example, raspy breath). The distortion threshold is set corresponding to a maximum level of distortion that is acceptable before the respiratory manoeuvre becomes obscured or corrupted by the distortion in the audio sample. The noise threshold is set corresponding to a minimum respiratory manoeuvre signal level that can be reliably differentiated from the background noise level in the environment of the user 106.

The optimum audio sample may be determined by selecting a specific audio sample from the audio sample dataset that meets the condition of having a signal-to-noise ratio above a noise threshold and/or a distortion level below a distortion threshold. In the event that more than one audio sample in the audio sample dataset meets the condition, the optimum audio sample may be selected as either: the audio sample in the audio sample dataset recorded at the closest distance from the user's mouth 107 meeting the condition or an audio sample in the audio sample dataset that was recorded further from the user's mouth 107 that has a higher audio quality (i.e., an improved trade-off between signal to noise ratio vs distortion). In this way, the accuracy of lung condition identification is improved because anomalous audio samples are eliminated. From the selected optimum audio sample, the corresponding distance (calculated as described above) between the smartphone 102 and the user's mouth 107 can then be selected as the optimum distance. Selecting a specific audio sample from the audio sample dataset in this way to identify the optimum distance is an efficient approach when the audio sample dataset contains a large number of audio samples, since the likelihood of the “true” optimum distance corresponding to the distance at which one of the audio samples in the audio sample dataset was recorded is relatively high.

Alternatively, the optimum audio sample may be predicted by mathematically fitting the signal-to-noise ratio and/or distortion level data of the audio samples in the audio sample dataset to predict a “virtual” optimum audio sample that would be expected to provide the highest signal-to-noise ratio and lowest distortion level. Once the “virtual” optimum audio sample has been predicted from the fit, a distance between the smartphone 102 and the user's mouth 107 can be determined that corresponds with the “virtual” audio sample. This “fitting” approach can be useful when the number of audio samples in the audio sample dataset is relatively low, as the “true” optimum audio sample (and corresponding “true” optimum distance) is more likely to fall somewhere between neighbouring audio samples recorded in the audio sample dataset.

Prior to step 304, the audio sample dataset may optionally be pre-processed, at the smartphone 102 or on a server, to identify an optimum period in each audio sample to be used in determining the optimum audio sample. The optimum period relates to a period of time in each audio sample which is best suited for identifying disease biomarkers. The optimum period may be based on a period of the audio sample having the highest signal-to-noise ratio. For example, there is typically a large peak in the amplitude of an audio sample of a forced exhalation during the first few seconds (for example, around three seconds) which can provide the greatest signal-to-noise ratio in that audio sample. In the event that a user performs a respiratory manoeuvre of a very short duration (e.g., less than three seconds), then a minimum noise level is identified in the associated audio sample to determine a minimum usable portion of the audio sample, and the time window of the audio sample in which that usable portion occurred may be used as the optimum period for that audio sample.

The optimum period may be determined for each audio sample on an individual sample-by-sample basis, or a common optimum period may be defined that is applied to all audio samples (for example, an average optimum period based on all audio samples in the present audio sample dataset, or an average optimum period across one or more previously recorded (i.e., historic) audio sample datasets). Once the optimum period has been determined, a signal-to-noise ratio and a signal distortion level is determined for each audio sample (step 304) from the optimum period of that sample.

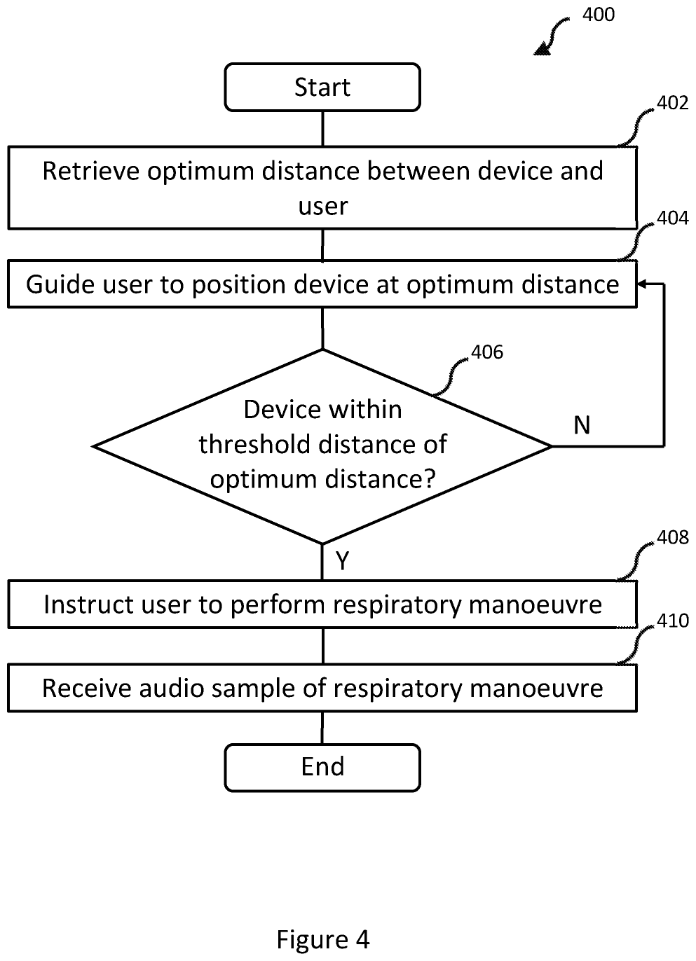

FIG. 4 illustrates a method 400 for recording an audio sample of a respiratory manoeuvre performed at the optimum distance 209.

At step 402, a smartphone 102 retrieves the optimum distance 209 (stored on the smartphone 102 or on a server).

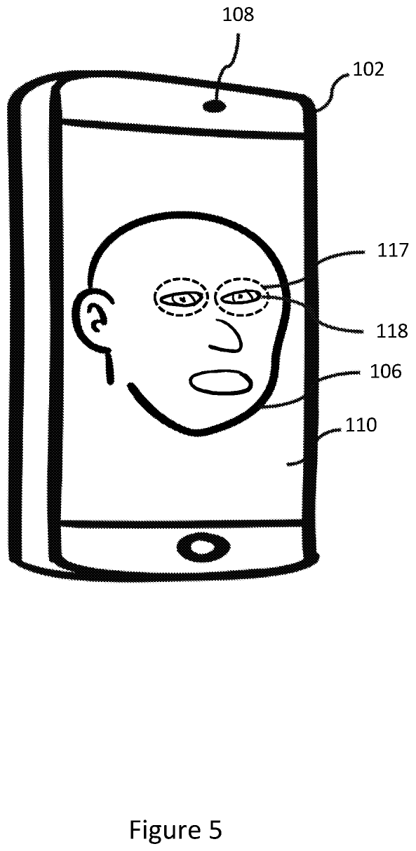

At step 404, the user 106 is guided to position the electronic device at the optimum distance 209 retrieved previously by either providing visual guidance on the user interface 110 (as shown in FIG. 5) and/or by audible guidance provided through a speaker of the smartphone 102.

At step 406, a determination is made whether the smartphone 102 is positioned within a threshold distance of the optimum distance 209. The current distance between the mouth of the user 106 and the smartphone 102 is determined from the pixel spacing between the eyes 118 of the user 106 in a live video feed from camera 108 using the relationship d=(W*f)/w, as discussed above. The current distance is compared with the optimum distance 209. If the current distance is not within a threshold distance of the optimum distance 209, the method returns to step 404 and the user 106 is guided to move the smartphone 102 closer to, or further from, their mouth 107 accordingly based on whether the current distance indicates that the smartphone 102 is too far away from, or too close to, their mouth 107. The threshold distance is based on a range either side of the optimum distance over which an acceptable audio sample of a respiratory manoeuvre for assessing lung function can be recorded.

Once the current distance is determined to be within the threshold distance of the optimum distance 209, the method proceeds to step 408 at which the user 106 is instructed to perform a respiratory manoeuvre at the optimum distance 209 by an indication on the user interface 110 on the smartphone 102 and/or an audible message provided to the user 106 through a speaker of the smartphone 102. The smartphone 102 receives an audio sample of the respiratory manoeuvre performed by the user 106 at the optimum distance 209 via an audio sensor in the smartphone 102 at step 410. The smartphone 102 may store the audio sample locally on a memory of the smartphone 102 and/or transmit the audio sample to a server. Analysis of the audio sample, such as determining one or more lung function measurements from the audio sample, may be carried out on the smartphone 102 and/or server.

FIG. 5 illustrates the smartphone 102 running a user interface 110 for guiding the user 106 to position their smartphone 102 at the optimum distance 209. The smartphone 102 overlays a mask 117 on top of a live video from the camera 108 showing the face of the user 106. The mask 117 guides the user 106 in locating their face at the optimum distance 209. In this example, the mask 117 is a pair of circles which are spaced according to the pixel spacing corresponding with a spacing between the eyes 118 of the user 106 expected at the optimum distance 209. The user 106 is guided to move the smartphone 102 forwards and/or backwards until the spacing between the eyes 118 of the user 106 in the live video occupies the mask 117. At this position, the face of the user 106 is at the optimum distance 209 ready for performing a respiratory manoeuvre. As well as this visual indication, audio feedback may be provided. For example, when the user 106 positions the smartphone 102 at the optimum distance 209, an audio tone may change in pitch and/or volume to alert the user 106 that the smartphone 102 is at the optimum distance 209.

### Guiding a User on the Optimum Mouth Shape for a Respiratory Manoeuvre

In conventional spirometry, a user seals their lips in an “O” shape around the end of a sample tube before performing a respiratory manoeuvre for use in assessing the lung function of the user. The mouth shape of the user is therefore controlled by the shape and size of the tube. In the absence of a tube, the user has no reference guide for the appropriate shape to make with their lips or how wide to open their mouth to perform the respiratory manoeuvre. If the surface area defined by the user's open mouth is smaller than an expected surface area of the user's throat, then the air flow produced by the user when performing an expiratory manoeuvre will be restricted, leading to inaccurate lung function data (e.g., the user's FEV1 may present as a lower value than the user is actually capable of, and a misdiagnosis may result).

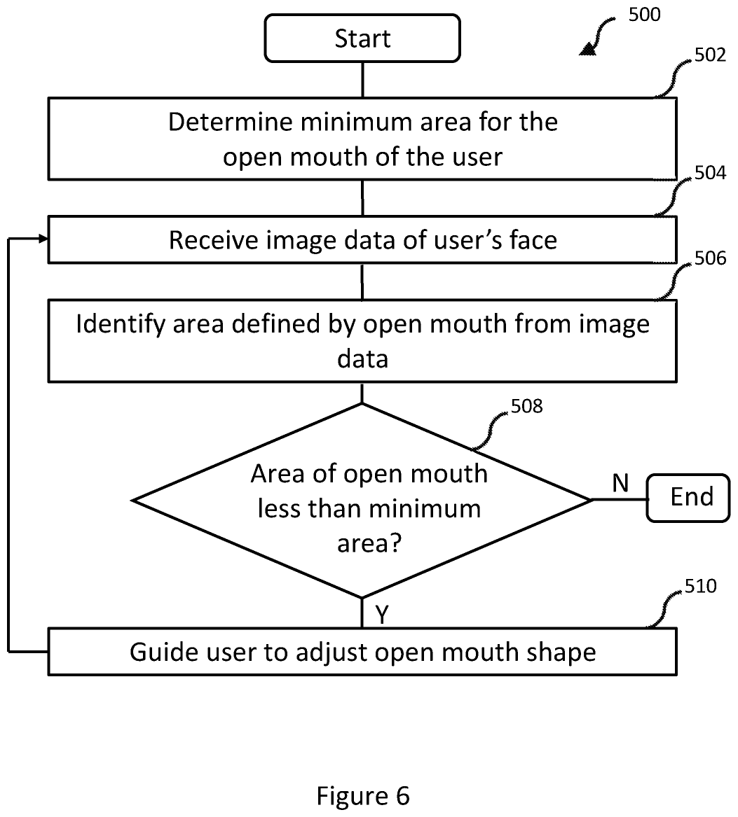

FIG. 6 is a flowchart illustrating a method 500 of determining an optimum open mouth shape of a user 106 providing an audio sample of a respiratory manoeuvre for use in assessing the lung function of the user 106.

At step 502, a minimum surface area for the open mouth of the user 106 is defined, for example, based on an expected area of the throat of the user 106. The minimum surface area is greater than or equal to the expected area of the throat 106 of the user 106, such that, substantially no air flow constriction occurs when the user 106 performs a respiratory manoeuvre. The expected area of the throat of the user 106 refers to an expected area of the user's pharynx or throat cavity, and is based on one or more of the age, gender and ethnicity of the user 106. The minimum area of the open mouth 107 may be obtained from a look-up table of expected values based on the characteristics of the user 106, such as their age, gender and ethnicity.

At step 504, a live video feed from the user-facing camera 108 in the smartphone 102 is obtained while the user 106 is guided to provide an open mouth shape. At step 506, an area defined by the open mouth of the user 106 is identified from the live video feed using an image processing algorithm to identify the position and size of the mouth 107 in the video images.

The position of the mouth 107 may be determined by first determining the position of the eyes 118 of the user 106 in the video images. The distance between human eyes is a well-characterised parameter of the human face that can be used to determine an approximate location and size of the mouth 107. For example, the location of the mouth 107 relative to the eyes 118 of the user 106 can be calculated as a ratio of the distance between the eyes of the user 106, e.g., the approximate location of the mouth 107 below the eyes 118 of the user 106 is 1.5 times the distance between the eyes 118 of the user 106.

Once the approximate location of the mouth 107 has been determined, the video images can be analysed to identify pixels in the region of the approximate location of the mouth 107 that correspond to the area of the video images occupied by the open mouth 107. Pixels that correspond to the open mouth would typically be darker than neighbouring pixels that correspond to, for example, the lips or chin of the user 106. For example, the image data can be analysed to identify pixels in the image that have a relative brightness that is below a brightness threshold. An exposure level of the camera 108 may be adjusted to account for features of the face of the user 106 such as skin tone, sunken eye sockets and facial hair such that pixels corresponding to the open mouth 107 have the lowest relative brightness in the image data. Once the total number of pixels below the threshold is determined, the approximate area of the open mouth 107 can be calculated using the number of pixels that fall below the threshold, using the estimated distance between the eyes 118 of the user to scale the image.

At step 508, the area of the open mouth 107 calculated in step 506 is compared with the minimum area determined in step 502 to determine whether the area of the open mouth 107 is at least equal to the minimum area. If the area of the open mouth 107 is determined to be less than the minimum area, the user 106 is guided at step 510 to adjust their open mouth shape until the area of their open mouth 107 is at least equal to the minimum area.

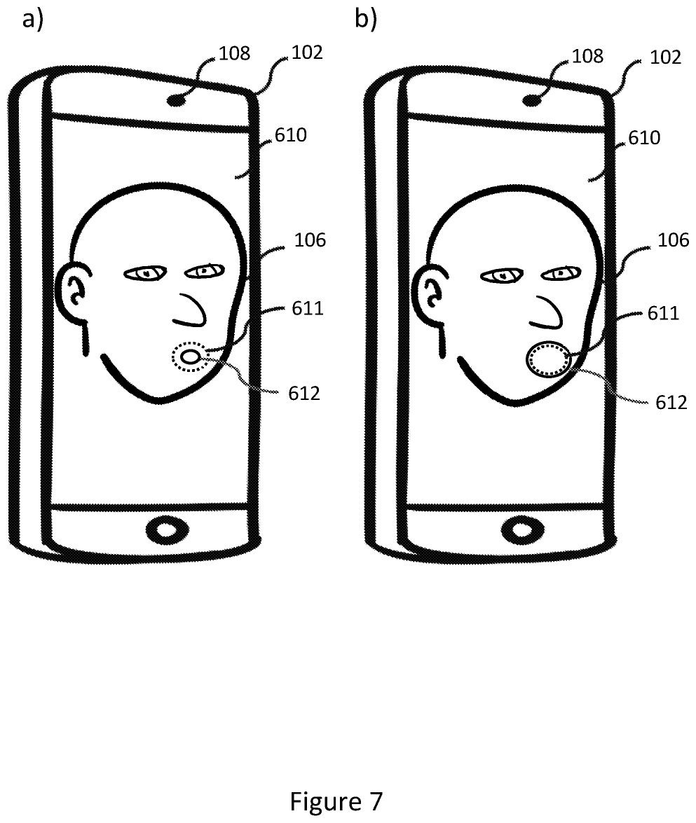

The user may be guided to adjust their open mouth shape until the area of their open mouth 107 is at least equal to the minimum area in a number of ways. In one example, FIG. 7 illustrates a user interface 610 for guiding a user 106 in providing an optimum mouth shape that is at least equal to the minimum area. The user interface 610 displays a live video feed from front-facing camera 108 showing the face of the user 106, including their mouth 612. An optimum mouth shape for the user 106 for providing a respiratory manoeuvre is determined according to the method of FIG. 6 and illustrated to the user 106 on the user interface 610 as an overlay 611. In FIG. 7a, the mouth 612 is smaller than the overlay 611, so the user 106 is guided by indications on the user interface 610 and/or audio feedback to adjust their mouth 612 to match their mouth to the shape of the overlay 611 (FIG. 7b), thereby obtaining a mouth shape that is at least equal to the minimum area.

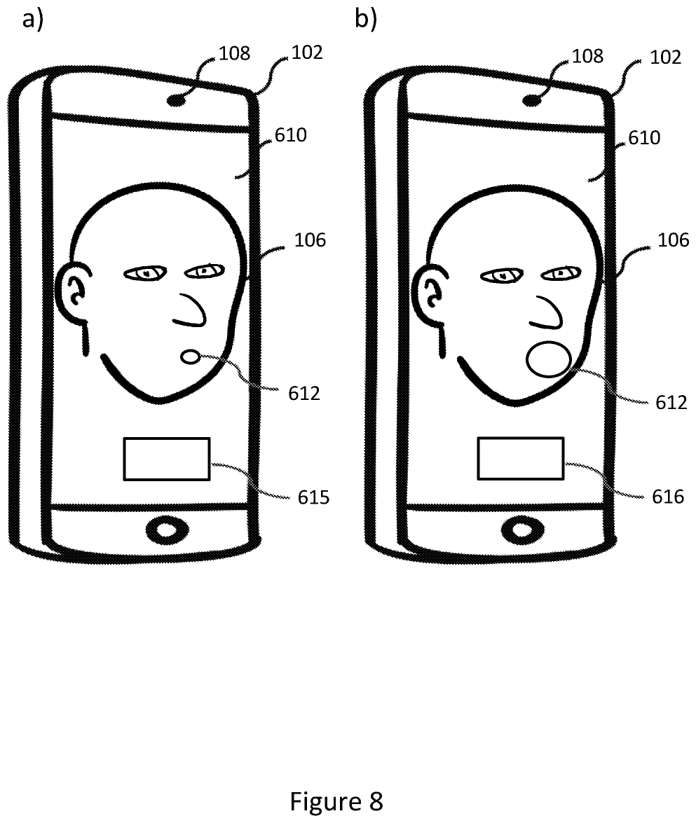

FIG. 8 illustrates a second example of a user interface 610 for guiding a user 106 in providing an optimum mouth shape that is at least equal to the minimum area. An optimum mouth shape for the user 106 for providing a respiratory manoeuvre is determined according to the method of FIG. 6 and illustrated to the user 106 on the user interface 610. However, rather than providing an overlay (as in FIG. 7), the user interface 610 instead presents a prompt to the user 106. In the example of FIG. 8a, the user's mouth 612 is smaller than the optimum mouth shape, and so a first prompt 615 is presented to the user 106 to indicate to the user to open their mouth 612 wider. Once the user 106 has opened their mouth 612 wide enough to meet the optimum mouth shape, the user interface 610 presents a second prompt 616 to the user 106 to indicate to the user that the optimum mouth shape has been achieved and to keep their mouth 612 at the current degree of openness. The first and second prompts 615, 616 may be text prompts, where the first prompt 615 presents a message to the user 106 to open their mouth 612 wider, and the second prompt 616 presents a message to the user 106 to maintain the openness of their mouth 612. Alternatively, the first and second prompts 615, 616 may form part of a traffic light system where the first prompt 615 is red light and the second prompt 616 is a green light.

Once the area of their open mouth 107 is at least equal to the minimum area (as in FIG. 7b or FIG. 8b), the user 106 is instructed to perform a respiratory manoeuvre by an indication on the user interface 610 on the smartphone 102 and/or an audible message provided to the user 106 through a speaker of the smartphone 102. The smartphone 102 receives an audio sample of the respiratory manoeuvre performed by the user 106 via an audio sensor in the smartphone 102. The smartphone 102 may store the audio sample locally on a memory of the smartphone 102 and/or transmit the audio sample to a server. Analysis of the audio sample, such as determining one or more lung function measurements from the audio sample, may be carried out on the smartphone 102 and/or server.

As discussed above, an optimum period may be determined for each audio sample. Following the initial determination as to whether the user 106 has obtained the optimum mouth shape discussed above with respect to FIGS. 6 to 8, the live video feed from the front-facing camera 108 may continue to be recorded to determine whether the user 106 has maintained the optimum mouth shape for at least a duration of the optimum period of each audio sample. For example, the recorded live video feed can be analysed, at the smartphone 102 and/or the server, to determine if the user 106 has maintained an optimum mouth shape that is equal to the minimum area for at least the first three seconds of the audio sample. This approach can be used as a means of “quality control” to determine whether a particular audio sample should be retained or discarded. For instance, if it is determined that the user 106 has maintained the optimum mouth shape for only part of, or none of, of the optimum period, then the corresponding audio sample can be classed as unacceptable and the user interface 610 can instruct the user 106 to maintain an optimum mouth shape and perform the respiratory manoeuvre until an acceptable audio sample is recorded.

It should be appreciated that the “quality control” process described above may be employed more broadly to determine whether a particular audio sample should be retained or discarded. For example, if the particular audio sample contains an audio signal suggestive of the user 106 performing a respiratory manoeuvre, but the corresponding recording of the live video feed indicates that the user's mouth 612 was closed for all or part of the audio sample, then it can be deduced that audio signal was likely due to background noise, rather than the user 106 performing the respiratory manoeuvre, and that the audio sample can be discarded. Additionally, if the particular audio sample contains a pronounced audio signal, but the corresponding live video feed recording indicates that the user's mouth 612 was open only briefly but then closed, this can be indicative that the audio signal recorded was due to the user 106 coughing, rather than performing the desired respiratory manoeuvre, and the audio sample can be discarded.

Additionally, by analysing an audio sample alongside a corresponding live video feed recording, it is possible to determine if an audio signal was produced by the user 106 performing the wrong kind of respiratory manoeuvre (for example, performing an inhalation when an exhalation was requested), for instance, by monitoring the live video feed recording for relative changes in a size of the user's head in the video images. As the user 106 performs an inhalation, they may move their head backwards and thus further away from the user interface 610, resulting in the size of their head in the video images reducing over a number of video frames. On the other hand, as the user 106 performs an exhalation, they may move their head forwards and thus closer to the user interface 610, resulting in the size of their head in the video images increasing over a number of video frames. Alternatively, or additionally, the video images of the live video feed may be monitored for changes in a facial expression of the user 106. For example, if the video images suggest the user is closing their mouth and/or puffing their cheeks, it may be determined that the user performed an inhalation. If the desired respiratory manoeuvre was an exhalation, but the user 106 is determined to have performed an inhalation, the user 106 can be prompted, via the user interface 610, to repeat the correct respiratory manoeuvre.

Although the invention has been described in terms of certain preferred embodiments, the skilled person will appreciate that various modifications could be made which still fall within the scope of the appended claims.

For example, although the invention has been described in terms of a smartphone, other personal electronic devices having audio and video capture could be used instead, such as a tablet device, or a desktop or laptop computer with an inbuilt camera or external webcam.

Whilst the invention has been described in terms of a user holding a smartphone in their hand at a distance from their mouth, for some users this it may not be appropriate or possible for the user to hold a smartphone in their hand. For example, some elderly users may have difficulties with holding a smartphone at different distances from their mouth. Also, users who suffer with shaking hands may experience difficulties holding a smartphone, and the shaking of their hands can corrupt the signal-to-noise ratio. Therefore, in some circumstances it may be more appropriate for a user to position the smartphone on a flat surface, such as a table, or propped up on another suitable surface. In this scenario, instead of being instructed to position the smartphone at a number of different distances from the user, the user may instead be instructed to position themselves at a number of different distances from the smartphone. Placing the smartphone on a flat surface may also be used for the process of guiding a user on an optimum mouth shape for a respiratory manoeuvre disclosed herein.

Whilst the process of measuring a distance between the smartphone and the user has been described as using images of the user (that is, based on the relationship between the physical distance between the eyes of the user, the pixel spacing between the eyes and the focal length of the lens of the camera), other techniques for measuring the distance between the smartphone and user could be used alternatively or additionally, such as by using a time of flight camera, IR sensor and/or LIDAR detector within the smartphone.

1. A computer-implemented method of obtaining an audio sample of a respiratory manoeuvre for use in assessing the lung function of a user, the computer-implemented method comprising:
determining a minimum area of an open mouth of the user required for providing an optimum audio sample of a respiratory manoeuvre for use in assessing the lung function of the user;
receiving, from a user-facing camera in an electronic device, image data of the user's face including at least an open mouth of the user;
identifying, from the image data, an area defined by the open mouth;
determining whether the area of the open mouth from the image data is at least equal to the minimum area of the open mouth required for providing the optimum audio sample of the respiratory manoeuvre; and
guiding the user to achieve the optimum open mouth shape, wherein guiding the user comprises providing an indication whether the area of the open mouth of the user is at least equal to the minimum area of the open mouth required for providing the audio sample of the respiratory manoeuvre;
wherein upon determining, from the image data, that the area of the open mouth is at least equal to the minimum area of the open mouth required for providing the audio sample of the respiratory manoeuvre, instructing the user to perform a respiratory manoeuvre; and
receiving, from an audio sensor in the electronic device, an audio sample of the respiratory manoeuvre.

2. The computer-implemented method of claim 1, further comprising:
receiving, from the user-facing camera in the electronic device, further image data of the user's face including at least the open mouth of the user as the user performs the respiratory manoeuvre; and
determining whether the minimum area of the open mouth is maintained for at least an optimum period of the audio sample.

3. The computer-implemented method of claim 1, wherein the indication provides either:
a prompt to the user on the electronic device, the prompt comprising a visual or audio cue for indicating to the user whether the minimum area of the open mouth required for providing the audio sample of the respiratory manoeuvre is achieved; or
an overlay on the image data displayed on the electronic device, wherein the overlay is based on the minimum area of the open mouth required for providing the audio sample of the respiratory manoeuvre.

4. The computer-implemented method of claim 1, wherein the minimum area of the mouth is related to an expected area of the throat of the user, for example, the minimum area of the mouth is greater than the expected area of the throat of the user.

5. The computer-implemented method of claim 1, wherein the area defined by the open mouth in the image is determined by segmenting the image into an open-mouth part and a non-open-mouth part by thresholding the image according to the relative brightness of the open-mouth part and the non-open-mouth part.

6. The computer-implemented method of claim 1, wherein the area defined by the open mouth in the image is determined by locating eyes in the image data and identifying the open mouth relative to the location of the eyes.

7. The computer-implemented method of claim 1, further comprising:
receiving, from the user-facing camera in the electronic device, further image data of the user's face including at least the open mouth of the user as the user performs the respiratory manoeuvre;
identifying, from the further image data, a size of the user's head; and
determining if the user is moving their head towards, or away from, the electronic device based on a change in the size of the user's head between successive frames of the further image data; and
based on the determination, determining if the respiratory manoeuvre is an expected respiratory manoeuvre.

8. A computer-readable medium, comprising instructions that when executed by a processor, cause the processor to carry out the method of claim 1.

9. A computer-implemented method of determining an optimum distance between an electronic device and a user providing an audio sample of a respiratory manoeuvre for use in assessing the lung function of the user, the computer-implemented method comprising:
receiving, from an audio sensor in the electronic device, an audio sample dataset comprising a plurality of audio samples of respiratory manoeuvres performed by the user at a plurality of distances between the electronic device and the user, wherein the audio sample dataset comprises at least one audio sample of a respiratory manoeuvre for each of the plurality of distances; and
determining an optimum distance for a user to provide an optimum audio sample of a respiratory manoeuvre for use in assessing the lung function of the user based on the audio sample dataset.

10. The computer-implemented method of claim 9, wherein the optimum distance is determined based on a signal-to-noise ratio and a distortion level for each of the plurality of audio samples in the audio sample dataset.

11. The computer-implemented method of claim 10, wherein either:
the optimum distance corresponds with an audio sample in the audio sample dataset having a signal-to-noise ratio above a noise threshold and/or a distortion level below a distortion threshold; or
the optimum distance corresponds with a predicted optimum audio sample based on the audio sample dataset, wherein the predicted optimum audio sample is determined by fitting a function to the signal-to-noise ratio and/or distortion level of the audio sample dataset in order to predict an optimum audio sample where the signal-to-noise ratio is maximised and the distortion level is minimised.

12. The computer-implemented method of claim 10, wherein determining the signal-to-noise ratio comprises receiving, from an audio sensor in the electronic device, background audio data relating to background noise of the environment of the user and comparing each audio sample with the background audio data.

13. The computer-implemented method of claim 9, further comprising:
receiving, from a user-facing camera in the electronic device, image data of the user's face corresponding with each of the audio samples;
extracting a feature of the user's face from the image data associated with each of the audio samples; and
determining a distance between the user and the electronic device of each audio sample based on the feature extracted from the image data associated with the respective audio sample;
optionally wherein the feature comprises a distance between the eyes of the user.

14. The computer-implemented method of claim 9, where the distortion level is based on the level of one or more of non-linear distortion, windshear, and clipping in an audio sample.

15. The computer-implemented method of claim 9, wherein the respiratory manoeuvre is an inspiratory manoeuvre or an expiratory manoeuvre; optionally wherein the optimum distance for an inspiratory manoeuvre is different to the optimal distance for an expiratory manoeuvre.

16. A computer-readable medium comprising instructions that, when executed by a processor, cause the processor to carry out the method of claim 9.

17. A computer-implemented method of recording an audio sample of a respiratory manoeuvre performed by a user for use in assessing the lung function of the user, the computer-implemented method comprising:
receiving, from an audio sensor in the electronic device, an audio sample dataset comprising a plurality of audio samples of respiratory manoeuvres performed by the user at a plurality of distances between the electronic device and the user, wherein the audio sample dataset comprises at least one audio sample of a respiratory manoeuvre for each of the plurality of distances;
determining an optimum distance for a user to provide an optimum audio sample of a respiratory manoeuvre for use in assessing the lung function of the user based on the audio sample dataset;
instructing the user to position the electronic device such that a distance between the user and the electronic device is within a threshold distance of the optimum distance and, upon determining that the distance between the user and the electronic device is within the threshold distance of the optimum distance, instructing the user to perform a respiratory manoeuvre; and
receiving, from an audio sensor in the electronic device, an audio sample of the respiratory manoeuvre.

18. The computer-implemented method of claim 17, wherein determining the distance between the user and the electronic device comprises:
receiving, from a user-facing camera in the electronic device, image data of the user's face;
extracting a feature of the user's face from the image data; and
determining the distance between the user and the electronic device based on the feature extracted from the image data; optionally wherein the feature comprises a distance between the eyes of the user.

19. The computer-implemented method of either of claim 18, wherein the respiratory manoeuvre is an inspiratory manoeuvre or an expiratory manoeuvre.

20. A computer-readable medium comprising instructions that, when executed by a processor, cause the processor to carry out the method of claim 17.
